(19) United States
(12) Patent Application Publication
(10) Pub. No.: US 2006/0045875 A1
(43) Pub. Date: Mar. 2, 2006

(54) Method of use of crotoxin as an anti-retroviral agent

(76) Inventor: Paul F. Reid, Plantation, FL (US)

Correspondence Address: Robert J. Van Der Wall;Colonial Bank Centre, Suite 1620, 1200 Brickell Avenue, Miami, FL, 33131, US

(21) Appl. No.: 10/934,594

(22) Filed: Sep. 2, 2004

(51) Int. Cl. A61K 38/48; C07K 14/47

(52) U.S. Cl. 424/94.63; 530/300

(57) ABSTRACT

The present invention provides a composition of matter and a method of using the composition for treating and preventing of retroviral infections of mammalian cells. It includes identification of Crotalus beta-neurotoxins as capable of preventing HIV infection and replication in that cell and a retroviral composition which can be administered in-vivo for the treatment of HIV infection. The retrovirus is selected from the group consisting of Lentiviruses (HIV-1, HIV-2, SIV, EIAV, BIV, and FIV). In addition, crotoxin can effect changes to the expression of HIV receptor proteins and/or cellular cofactors homologous to their target receptors.

### BACKGROUND OF THE INVENTION

1. Field of the Invention

The present invention relates to a composition of matter involving a class of proteins and a method of use of that composition for treatment of viral diseases. It is especially directed to the treatment of heretofore intractable diseases such as retro-viral infections, including specifically HIV infections.

2. Description of Prior Art

Venoms have been reportedly employed as antiviral and analgesic agents prior to World War II (Clarke and Baldone, 1962). U.S. Pat. No. 3,657,416 discloses an enzyme isolated from the venom of the snake Agkistrodon rhodostoma and the use of this enzyme for the therapeutic treatment of humans. Sanders, et al. had commenced investigating the application of modified venoms to the treatment of ALS in 1953 having employed poliomyelitis infection in monkeys as a model. Others antiviral studies had reported inhibition of pseudorabies (a herpesvirus) and Semliki Forest virus (alpha-virus). See Sanders' U.S. Pat. Nos. 3,888,977, 4,126,676, and 4,162,303.

Miller, et al. (1977) reported that the modified venoms antiviral activity against Semliki Forest virus was associated with several chromatographic fractions comprising the neurotoxic components. The most abundant component with antiviral activity was shown to be alpha-cobratoxin. Yourist, et al. (1983) reported that modified alpha-cobratoxin could inhibit the activity of herpesvirus while the native toxin was ineffective.

U.S. Pat. No. 4,341,762 discloses compositions having pharmacological activity and which contain, in an administrable form, at least one post-synaptic neurotoxin, at least one pre-synaptic neurotoxin, and at least one component capable of stimulating the immune mechanisms of the body. The post-synaptic neurotoxin component preferably contains the .alpha.-toxin obtained from the venom of an elapid snake belonging to a species of the genera Naja, Ophiophagus or Dendroaspis. The pre-synaptic neurotoxin component preferably contains .beta.-bungarotoxin obtained from the venom of the elapid snakes Bungarus multicinctus and the component capable of stimulating the immune mechanisms of the body preferably contains a venom obtained from a viperid snake, particularly a venom obtained from a snake belonging to the family Vipera, subfamily Crotalinae.

Research to determine the composition of and the specific effect produced by the individual components of snake venoms has been ongoing. It has been found that snake venom contains a plurality of proteinaceous and other components or substances. Certain snake venoms or fractions thereof have exhibited therapeutic activity as anticoagulants and as analgesics.

The compositions disclosed in U.S. Pat. No. 4,341,762 may include the whole venom from which a particular activity is being sought or only a particular fraction or fractions of the venom. Thus, the pre-synaptic neurotoxin may consist of the whole venom of a Bungarus species. A whole venom from the Naja species contains post-synaptic neurotoxins which can potentially complement the pre-synaptic activity of the Bungarus component of the mixture. The component capable of stimulating the immune mechanisms of the body, the viperid component, is preferably used and is present as the whole venom since such venoms contain a plurality of enzymatic substances.

The compositions disclosed in U.S. Pat. No. 4,341,762 stimulate the production of the substance known as “interferon” or a precursor thereof. The compositions set forth in U.S. Pat. No. 4,341,762 are useful as antiviral and anti-autoimmune agents by the stimulation of the part of the body's immune system which involves interferon or the complex activity within the body attributed to interferon. The compositions disclosed in U.S. Pat. No. 4,341,762 can be used in the treatment of the symptoms of poliomyelitis, herpes simplex, herpes zoster, herpes genitalis, and degenerative neurological disorders.

The compositions set forth in U.S. Pat. No. 4,741,902 are an improvement of those disclosed in U.S. Pat. No. 4,341,762. These compositions also contain effective amounts of at least one post-synaptic neurotoxin and at least one pre-synaptic neurotoxin, but the component capable of stimulating the immune mechanisms of the body is the viperid “b” fraction obtained from elution of the viperid venom on SEPHADEX.

Valdes et al., (U.S. Pat. No. 5,676,974) report the use of a complex to treat HIV infections comprising gyroxin and a phospholipase protein (3.1.1.4). The phospholipase referred to in this patent is that of the B subunit of crotoxin. It is claimed that a number of individuals treated with this composition were clinically cured. However, the mechanism by which the composition can treat AIDS remains obscure. No pharmaceutical action is ascribed to gyroxin, a thrombin-like peptide. Phospholipases are isolated from a number of sources and readily disrupt the cellular membranes of animals, gram negative bacteria and, presumably, those of enveloped viruses of which HIV is one. However, in the absence of the A subunit (crotapotin), the phospholipase subunit is deemed non-specific in its activity as related by Plata et al (U.S. Pat. No. 5,164,1961).

Most recently, several scientific communications suggested the potential use of venom constituents in the inhibition of HIV. Fenard et al. showed secreted phospholipases inhibited HIV replication. More significantly snake venom phospholipases inhibited HIV replication. It was found that the phospholipase activity was not required for inhibition. It was also notable that the snake venom products with the greatest antiviral activity were single-chain enzymes with the exception of Taipoxin, which is a three-subunit enzyme.

Other patent references of interest include MacDonald et al., U. S. Pat. No. 5,723,477. Literature references of interest are: Bracci L., Lozzi L., Rustici M. and Neri P.; FEBS 311:115-118 (1992), Choe H., Farzan M., Sun Y., Sullivan N., Rollins B., Ponath P. D., Wu L., Mackay C. R., LaRosa G., Newman W., Gerard N., Gerard C. and Sodroski J.; Cell 85:1135-1148 (1996), Clarke, W. B., Baldone, J. A. and Thomas, C. I., South. Med. J., 55, No. 9, 947-951 (1962), Costa, L. A., Miles, F, Diez, R. A., Araujo, C. E., Coni Molina, C. M. and Cervellino, J. C.; Anticancer Drugs 8 (9), 829-34 (1997), Courgnaud V., Pourrut X., Bibollet-Ruche F., Mpoudi-Ngole E., Bourgeois A., Delaporte E. and Peeters M.; J. Virol. 75:857-866 (2001), Cura, J. E., Blanzaco, D. P., Brisson, C. B., Cura, M. A., Cabrol, R., Larrateguy, L., Mendez, C., Sechi, J. C., Silveira, J. S., Theiller, E., deRoodt, A. R., and Vidal, J. C.: Clinical Cancer Research, Vol. 8, 1033-1041 (2002). De Clerque E.; Mini. Rev. Med. Chem. 2:163-175 (2002), Delot, E., & Bon, C., J. Neurochem. 58, 311-319, (1992) Deng H., Liu R., Ellmeier W., Choe S., Unutmaz D., Burkhart M., diMarzio P., Marmaon S., Sutton R. E., Hill C. M., Davis C. B., Peiper S. C., Schall T. J., Littman D. R. and Landau N. R.; Nature 381:661-666 (1996), D'Souza M. P., Cairns J. S. and Plaeger S. F.; J.A.M.A. 284:215-222 (2000), Fenard et al. J. Clin. Invest, 104, No. 5, pg 611-168 (1999), Feng Y., Broder C. C., Kennedy P. E. and Berger E. A.; Science 272:872-877 (1996), Greenhead P., Hayes P., Watts P. S., Laing K. G., Griffin G. E. and Shattock R. J.; J. Virol. 74:5577-5586 (2000), Harvey, A. and Karlsson, E., Br. J. Pharmacol. Sep; 77(1):153-61 (1982), Jiang S., Zhao Q. and Debnath A. K.; Curr. Pharm. Des. 8:563-580 (2002), Korber B., Muldoon M., Theiler J., Gao F., Gupta R., Lapedes A., Hahn B. H., Wolinsky S., Bhattacharya T.; Science 288:1787-1796 (2000), Lambeau, G., Barhanin, J., Schweitz, H., Qar, J., and Lazdunski, M., J. Biol. Chem. 264(19), 11503-11510 (1989), Lentz T. L., Burrage T. G., Smith A. L., Crick J. and Tigor G. H.; Science 215:182-184 (1982), Lentz T. L., Hawrot E. and Wilson P. T.; Proteins:Structure, Function and Genetics 2:298-307 (1987), Miller K. D., Miller G. G. and Sanders M., Fellows O. N.; Biochem. Biophys. Acta 496:192-196 (1977), Moore J. P., Sattentau Q. J., Wyatt R. and Sodroski J.; J. Virol. 68:469-484 (1994), Neri P., Bracci L., Rustici M. and Santucci A.; Arch. Virol. 114:265-269 (1990), Oshima-Franco, Y., Leite, G. B., Silva, G. H., Cardoso, D. F., Hyslop, S., Giglio, J. R., da Cruz-Hofling, M. A., and Rodrigues-Simioni, L. Toxicon, 39(10), 1477-85 (2001), Patterson, B., Flener, Z., Yogev, R. and Kabat, W. “Inhibition of HIV-1 replication in mononuclear cells and thymus explant cultures by a purified, detoxified cobra venom protein” (2000) Abstract, “Novel biological fusion inhibitors of HIV”, Apr. 7, 2000, Keystone Conference, Colorado., Peters B. S.; Antivir. Chem. Chemother. 11:311-320 (2000), Sanders, M., Soret, M. G. and Akin, B. A.; Ann. N. Y. Acad. Sci. 53: 1-12 (1953), Soares, A. M., Guerra-Sa, R., Borja-Oliveira, C. R., Rodrigues, V. M., Rodrigues-Simioni, L., Rodrigues, V., Fontes, M. R. M., Lomonte, B., Gutiérrez, J. M. & Giglio, J. R., Archives of Biochemistry and Biophysics, 378: 201-209 (2000), Starcich B. R., Hahn B. H., Shaw G. M., McNeely P. D., Modrow S, Wolf H., Parks E. S., Parks W. P., Josephs S. F. and Gallo R. C.,; Cell 45:637-648 (1986), Sullivan N., Sun Y., Sattentau Q., Thali M., Wu D., Denisova G., Gershoni J., Robinson J., Moore J., and Sodroski J.; J. Virol. 72:4694-4703 (1998), Thali M., Moore J. P., Furman C., Charles M., Ho C. C., Robinson J. and Sodroski J.; J. Virol. 67:3978-3988 (1993), Turpin, J. A. Expert. Opin. Investig. Drugs, 11 (8), 1077-1097 (2002), VanDamme L., Wright A., Depraetere K., Rosenstein I., Vandermissen V., Poulter L., McKinlay M., Van Dyck E., Weber J., Profy A., Laga M. and Kitchen V.; Sex. Transm. Infect. 76:126-130 (2000), Wu L., Gerard N. P., Wyatt R., Choe H., Parolin C., Ruffing N., Borsetti A., Cardoso A. A., Desardin E., Newman W. and Sodroski J.; Nature 384:179-183 (1996), Wyatt R., Moore J., Accola M., Desjardin E., Robinson J. and Sodroski J.; J. Virol. 69:5723-5733 (1995), Yourist, J. E., Haines, H. G. and Miller, K. D., J. Gen. Virol 64:1475-1481 (1983).

### SUMMARY OF THE INVENTION

The present invention provides a composition and method for treating and preventing retroviral infections of mammalian cells. One aspect of the invention relates to the identification of Crotalus beta-neurotoxins capable of preventing HIV infection and replication in that cell. In another aspect the invention relates to an retroviral composition which can be administered in-vivo for the treatment of HIV infection. In another aspect, the retrovirus is selected from the group consisting of Lentiviruses (HIV-1, HIV-2, SIV, EIAV, BIV, and FIV)

Proteins such as those from venoms, as described herein, have long been recognized for their ability to bind to specific receptors on the surface of mammalian cells. These neurospecific proteins bind to such common receptors as the acetylcholine receptor for example. Costa et al. (1997) and Cura et al. (2002) provide methods that allow the safe administration of crotoxin permitting the application of these laboratory observations to practical use. Therefore included in the invention is a method of treating lentivirus infection in mammals and humans comprising administering the neurotoxin to the host.

In yet another aspect of the invention is the indication that crotoxin can affect changes to the expression of HIV receptor proteins and/or cellular cofactors homologous to their target receptors.

In another aspect of the invention, crotoxin's antiviral activity suggests the possibility of synergism with other neurotoxic venom components due to the potential for similar mechanisms displayed by other neurotoxic components. Alpha-neurotoxins, such as cobratoxin, also with acetylcholine receptor blocking activity can inhibit lentivirus infection. Furthermore, cardiotoxin's lytic capabilities may enhance the antiviral effect of crotoxin similarly for that described by Vidal (U.S. Pat. No. 5,232,911) in crotoxin's anticancer application.

### DETAILED DESCRIPTION OF THE INVENTION

Although the survival of individuals currently infected by the HIV virus is dramatically longer than it was 20 years ago, such survival is at the cost of a drug regime which is highly expensive, complicated, relegated to a fixed time and sequence schedule, has adverse physiological side effects and is, ultimately, too little too late. While the logical method to halt the spread of the disease is sexual abstinence, such a method embodies so many facets of world society, that, realistically, the disease will remain uncontrollable until such a time as it can be controlled by methods which are inexpensive, have few side effects, and can be administered easily.

Prophylaxis, utilized before or after potential exposure, fulfills these requirements. Potential prevention/treatment could take many forms. Three are:
1. The development of a vaccine that prevents infection;
2. Prevention of an initial infection or control of the spread of an initial infection that has not progressed to AIDS by a means other than a vaccine; or
3. A resolution of the syndrome known as AIDS by the use of anti-retroviral agents.

While vaccine production is ultimately the most efficacious of the three methods, due to the mutational idiosyncrasies of the virus, such a development is not a likely or a probable near term occurrence. Vaccine development attempts to date have failed to translate into man from animal test-models (Peters; 2000).

Many medical research resources are currently being applied to the management, rather than the cure, of HIV infection. While the use of antiretrovirals have improved the quality and length of life, they have disadvantages which include toxicity, development of drug resistance, persistence of latently infected cells resulting in viral rebound after prolonged treatment and, finally, high expense. The prevention and/or control of an infection prior to loss of immune capabilities associated with progression to AIDS is currently the most expedient and cost effective method. Currently, there are several approved drugs types that apply themselves to the control of an ongoing HIV infection. These drug types are nucleoside reverse transcriptase inhibitors, non-nucleoside reverse transcriptase inhibitors and protease inhibitors. These are currently encompassed by highly aggressive anti-retroviral therapy (HAART). All these drug types are susceptible to loss of effectiveness due to genetic mutation of the HIV-1. Thus, the blockade of HIV infection or the control of the spread of HIV infection through the use of fusion or entry inhibitors appears to be the most logical method barring the availability of a vaccine. Such blocking substance, or substances, could be applied topically, as a cream or douche, and provide protection during coitus. The use of this mode of prevention has been suggested by others (Turpin, 2002) and is being implemented (Van Damme, et al., 2000). The utilization of a binding/entry inhibitor as a prophylactic that would block infection and maintain a period of protection in the genital tract could provide an effective measure which would reduce HIV-1 transmission (D'Sousa, et al., 2000). Topical administration would not be amenable to prevention of disease by blood transfer by more direct routes (such as hypodermic needles). However, as an injectable, or by buccal administration, it could be applicable parenterally in the treatment of an HIV infection during early stages of exposure, or later, by providing control of HIV dissemination within the host.

HIV-1 is a lentivirus (lenti=slow {Latin}) of the family Retroviridae. The virus is enveloped, 80-130 nm in diameter and has an icosahedral capsid. As with other lentiviruses, HIV can infect terminally differentiated, non-dividing cells such as macrophages resident in tissue or brain (microglia) as well as cells of the T cell lineage, specifically CD4+ cells, known as T helper (T_{H}) cells. Lentiviruses have, through mutation, the capability to infect immune cells (macrophages; T_{H}-cells), the ability to avoid immune system eradication and, thus, tend to persist for the life of their host. The typical HIV infection progresses through three stages: initial, or acute, associated with high levels of viral replication and dissemination, a latent stage attributed to partial immune system control, which is followed by the third stage which encompasses the return of high levels of viral replication and clinical disease, termed acquired immunodeficiency syndrome (AIDS). HIV is suggested to be derived from the simian immunodeficiency virus (SIV) (Courgnaud, et al., 2001) and first entered the human population between 1915 and 1941 (Korber, et al., 2000). Two HIVs are associated with human AIDS: HIV-1 and HIV-2. HIV-1 is distributed worldwide and is responsible for the current AIDS pandemic while HIV-2 is currently restricted to West Africa. Both are spread by the same routes, though HIV-2 may be less pathogenic.

Treatment of HIV infection currently encompasses two basic modalities: drug action at host intracellular targets (post entry) and drug interaction at viral extracellular targets (pre-entry). The latter are termed as binding/entry inhibitors. Extracellular targets are those associated with viral attachment, fusion and entry into the host cell. Intracellular targets are those associated with viral nucleic acid synthesis and processing and are termed as anti-retroviral drugs. There are currently 16 licensed antiretroviral drugs employed to combat HIV-1 infection (D'Souza, et al. 2000). Currently, there are no licensed drugs that are binding/entry inhibitors. Within the context of this application, extracellular targets are of immediate importance. Consequently, description of viral inhibition post-cell entry is omitted.

Infection by HIV occurs following the introduction of the virus to the blood of the potential host. Virus-host cell interaction is mediated through the viral envelope glycoproteins gp120 and gp41 (gp160), which are assembled as trimers on the surface of the viral envelope, and their interactions with host cell surface receptors CD4, and CXCR4 or CCR5. U.S. Pat. No. 5,994,515 (Hoxie) describes the manner in which the human immunodeficiency viruses HIV-1 and HIV-2 and the closely related simian immunodeficiency viruses (SIV), all use the CD4 molecule as a receptor during infection though viruses like HIV and FIV can infect CD4 negative cells. The latter two host cell surface receptors are chemokine receptors and act as co-receptors along with CD4. Chemokines are a large family of low molecular weight, inducible, secreted, proinflammatory cytokines which are produced by various cell types. See, for instance, Au-Yuong, et al., U.S. Pat. No. 5,955,303. Chemokines have been divided into several subfamilies on the basis of the positions of their conserved cysteines. The CC family includes monocyte chemoattractant protein-1 (MCP-1), RANTES (regulated on activation, normal T cell-expressed and secreted), macrophage inflammatory proteins (MIP-1.alpha., MIP-1.beta.), and eotaxin. (Proost, P. (1996) Int. J. Clin. Lab. Res. 26: 211-223; Raport, C. J. (1996) J. Biol. Chem. 271: 17161-17166). The CXC family includes interleukin-8 (IL-8), growth regulatory gene, neutrophil-activating peptide-2, and platelet factor 4 (PF-4). Although IL-8 and PF-4 are both polymorphonuclear chemo-attractants, angiogenesis is stimulated by IL-8 and inhibited by PF-4. However, the macrophage tropic (CCR5) strain BaL, is not capable of infecting cells which co-express both CXCR4 and CD4. These results suggest that CXCR4 can serve as a co-factor for T-tropic, but not M-tropic, HIV-1 strains (Feng, et al., 1996, supra). Moreover, the finding that there is a change from M to T-tropic viruses over time in infected individuals correlates with disease progression, and suggests that the ability of the viral envelope to interact with CXCR4 represents an important feature in the pathogenesis of immunodeficiency and the development of full blown AIDS.

There are five variable regions and five conserved regions that compose gp120 (Starcich, et al., 1986; Wyatt, et al., 1995). Two variable loop regions, V1/V2 and V3, prior to initial viral interaction with the cell surface, are closely associated and block accessibility to a region associated with chemokine receptor binding. Binding of CD4, which occurs above these two variable regions, is dependent upon discontinuous elements in conserved regions 3 and 4 (C3 and C4) (Moore, et al., 1994). Amino acid changes in the V2 and V3 loop regions can alter both the membrane fusion process and HIV-1 tropism (Wyatt, et al., 1995).

Infection of susceptible cells occurs via three conformational stages involving HIV-1 gp120 (D'Sousa et al., 2000). In short, the interaction between HIV-1 and the host cell proceeds as follows: A segment of gp120 binds to CD4 on the host cell surface resulting in an initial conformational change of the V1/V2 and V3 regions of gp120. This change allows access to a portion of gp120, previously covered by the two variable regions, which binds with a co-receptor resident on the host cell. This gp120 conformational change involves movement of the V1/V2 loops away from the V3 loop (Thali, et al., 1993; Wyatt, et al., 1995, Sullivan, et al., 1998). Under normal circumstances, HIV-1 gp120 requires the presence of both the CD4 and a co-receptor to cause additional conformational changes resulting in exposure of gp41. The protein, gp41, is responsible for fusion and entry. This co-receptor is either CXCR4 or CCR5 and is determined by the tropism of the virus (Feng, et al., 1996; Deng, et al., 1996; Choe, et al., 1996; Wu, et al., 1996). The extracellular portion of gp41 contains two helical domains: HR1 and HR2 (or NHR and CHR; Jiang, et al., 2002). The tip of gp41 inserts into the host cell membrane and anchors the virus to the cell. The two helical domains of gp41, previously separated by a segment of gp120, bind together to form a 6-helix bundle that is a fusogenic structure (Jiang, 2002). The virus and cell surface are pulled together by this structure, allowing fusion of the virus envelope and host cellular membrane and insertion of viral genetic material. The co-receptor CCR5, whose natural ligands are the α chemokines RANTES, MIP-1-α, MIP-1-β and MDC, is employed by primary isolates of HIV-1 which are generally M (macrophage) tropic, and are found on T cells and macrophages. CXCR4, whose natural ligand is SDF-1α, is employed by late stage HIV-1 isolates and is employed by T (T cell)-tropic HIV-1. There is an in vivo switch in tropism during HIV infection (Wyatt and Sodroski, 1998).

Due to the complexity of the binding and penetration of HIV-1, the virus is, at least theoretically, vulnerable to either single or, more especially, multiple entry inhibitors. Therefore, there are several cellular sites and viral sites with which inhibitors could interact to halt the process: CD4, CXCR4, CCR5, gp120 and gp41. The substances currently under consideration generally have high cost in addition to limited production as well as low bio-availability and poor pharmacologic and toxicology profiles. Nineteen potential binding/entry inhibitors were listed in 2000 (D'Sousa, et al., 2000); work is still progressing and a glance at the current literature indicates new additions in the list. Gp41 inhibitors T-20 and T-1249 (Trimeris/Hoffman LaRoche) as well as PRO-542 (Progenics), PRO-2000 (Procept) and Cyanovirin (CV-N) all of which target virus/CD4 interaction and AMD-3100 (AnorMed), which interferes with HIV/CXCR4 interactions, are still viable candidates. These compounds are representative of, and provide an overview of, current thought in the area of inhibiting viral binding/entry (De Clercq, 2002).

The drug candidates listed above suggest that combinatorial efforts to prevent binding and entry is likely to become the norm, as opposed to the use of single drugs, as indicated by the synergistic combination of drugs with T-20. Additionally, the concept of disease prevention by the use of binding/entry inhibitors is established in the research and clinical communities. The use of PRO-2000 in a vaginal gel, coupled with the early results achieved, suggest that this is a potentially viable approach, especially given that this is associated with the most frequent mode of transmission (Greenhead, 2000). This topical approach is strengthened by the determination that HIV must transit the epithelial lining of the vagina wall to access infection susceptible cells, that epithelial cells are not subject to infection and they do not aid transport of the virus. In fact, the epithelial cells may act as a barrier to infection. The presence of PRO 2000 was found to result in 97% reduction in HIV infection in an in-vitro cervical explant test system (Greenhead, 2000).

Cobra venom components have been demonstrated to inhibit HIV replication (Patterson et al., 2000). Death by cobra envenomation is attributed to the interaction of basic polypeptides (cobra alpha-neurotoxins) that act post-synaptically and result in blockade of nerve transmission due to their affinity for the nicotinic acetylcholine receptor (nAchR). nAchRs are ligand-gated ion channels activated by the binding of acetylcholine (Ach). Cobratoxin and other snake alpha-neurotoxins are curaremimetic since they mimic the actions of curare in that they are potent competitive inhibitors of Ach binding to the nAchR and blocking Ach activity. A large number of curaremimetic toxins have been isolated from the venoms of elapid and hydrophid snakes and similar curaremimetic toxins have been isolated from the venom of sea snails of the Conus genera.

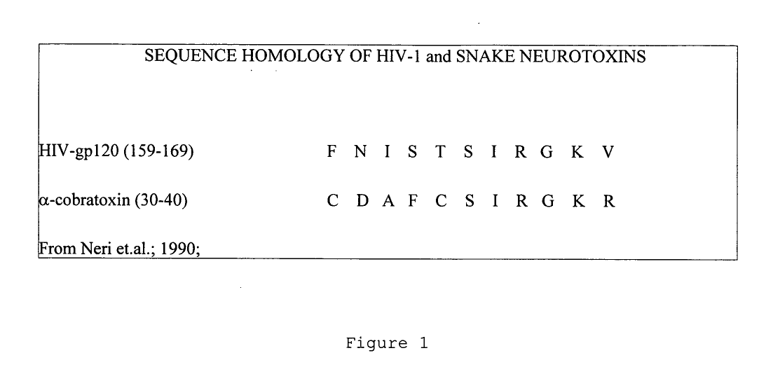

The α-neurotoxins of Naja naja kaouthia (cobratoxin) and Bungarus multicinctus (bungarotoxin) have a sequence homology with HIV gp120. See FIG. 1. Like the homologous sequence on elapid toxins, the amino acid sequence present in rabies virus glycoprotein (RVG) and gp120 of HIV results in interaction with the nAchR. This interaction has been demonstrated by the binding of rabies virus (Lentz, et al., 1982, Lentz, et al., 1987) and HIV-1 gp120 (Bracci, et al., 1992). Both viral interactions were blocked by the use of α-bungarotoxin.

The ability of HIV-gp120 to bind to the nAchR as well as the proven capability of alpha-neurotoxins to bind to the same receptor permits the hypothesis that neurotoxins may act as an entry inhibitor particularly in the nervous system.

Crotoxin is a non-covalent complex having a molecular weight of 24 kD and is formed by two non identical subunits: a basic one (crotoxin subunit B, molecular weight 14.5 kD) and an acidic one (crotoxin subunit A, molecular weight 9.5 kD). Crotoxin subunit A is non-toxic and devoid of catalytic activity. Subunit A is formed by three polypeptide chains A, B and C cross-linked by seven disulfide bonds. When properly aligned, the polypeptide chains (A, B and C) exhibit sequence similarities with other non-toxic phospholipases. Isoforms of subunit A which differ in two or three amino acid residues at the beginning and at the end of chain A appear to be generated by the proteolytic cleavage of a precursor polypeptide homologous to a phospholipase A₂ (Bon, 1997). The structure and production is described in detail in the specifications of the following patents: U.S. Pat. No. 5,164,196 and U.S. Pat. No. 5,232,911.

It has been previously shown that iodinated crotoxin and taipoxin bind specifically with high affinity to the isolated synaptic membrane fraction from guinea-pig brain, whereas specific binding is not detected with the nontoxic pancreatic phospholipase A2. Experiments based on photoaffinity labeling and simple chemical cross-linking techniques have led to the identification of three polypeptides preferentially present in neuronal membranes as (subunits of) the binding protein(s) for crotoxin and/or taipoxin. Some, but not all, other toxic phospholipases A2 also appear to be ligands for the three polypeptides. It has been found that under Ca(2+)-free condition, taipoxin or crotoxin inhibit with a 50% inhibition constant (IC50) of 20-1000 nM the Na(+)-dependent uptake of norepinephrine, dopamine and serotonin by the synaptosomes. In contrast, choline uptake is not affected.

Crotoxin is known to desensitize the nicotinic receptor of Torpedo marmorata and Electrophorus electricus electroplaques. It has been found that the purely cholinergic synaptosomes from the Torpedo electric organ provided a convenient model to study the pharmacology of crotoxin and other related neurotoxins (Delot, E., & Bon, C., 1992). Labeled 125I crotoxin demonstrated saturable binding to Torpedo presynaptic membranes. In the range of concentrations that was effective on synaptosomes, crotoxin bound to a single class of sites without cooperativity. 4-Aminopyridine antagonism of the crotoxin-induced blockade of the end-plate depolarization produced by carbachol show that the postsynaptic effect of crotoxin at the guinea-pig muscle end-plate also results from nicotinic receptor desensitization.

Therefore, the purported dual mechanism of crotoxin suggests that the anti-retroviral effect could stem from either the impairment of acetylcholine release or nicotinic acetylcholine receptor desensitization. While there is no obvious homology between cobratoxin and crotoxin, they display similar pharmacological actions. Desensitization by crotoxin of the nicotinic receptor would mimic the effects described for cobratoxin suggesting it could elicit antiviral effects.

Fenard et al. (1999) proved that certain venom phospholipases could inhibit the replication of HIV in Peripheral Blood Mononuclear Cells (PBMCs). The highest activity was observed in those phospholipases isolated from snakes such as elapids, all of which were neurotoxic. The catalytically inactivated Viper phospholipase showed poor activity in the HIV assay though an active phospholipase activity was reported not to be involved in HIV inhibition. This might suggest that the preferred phospholipase for use in anti-HIV assays should be derived from cobras. Perhaps the inactivated viper component missing the correct structural determinant compared to deactivated cobra phospholipases. This myotoxin phospholipase from Bothrops asper was not reported to be neurotoxic. The venom of Bothrops neuwiedii is reported to contain a neurotoxic phospholipases (Soares et al, 2000) and the neurotoxic component in Bothrops jararacussu venom can be neutralized by crotoxin antibodies (Oshima-Franco et al., 2001). With the exception of taipoxin, all the phospholipases examined were also single chain peptides. Taipoxin is a tri-peptide complex. Taipoxin and crotoxin display poor phospholipase activity when covalently linked to their other subunit(s)(Lambeau et al., 1989). Taipoxin's and crotoxin's presynaptic activity are distinct (Harvey and Karlsson, 1982). Given the neurotoxic similarities of crotoxin to those employed in the Fenard et al. study, in addition to the desensitizing activity of crotoxin at the nicotinic receptor it was logical to confirm the potential for crotoxin as an inhibitor of HIV replication and which specific subunit comprising crotoxin manifests this activity if the complex was ineffective or active.

Production Techniques

Methods for the purification of crotoxin have been described in U.S. Pat. No. 5,164,196. A precipitation step can be employed to assist in the removal of extraneous proteins simply by re-suspending the venom in a high pH, low ionic strength solution and adjusting the pH to between 6-7. Crotoxin will efficiently precipitate under these conditions. The precipitate can be washed and dissolved again by adjusting the pH to over 9.5. The resulting crotoxin complex could be employed as a therapeutic at this point, certainly for veterinary use. As the crotoxin complex is required intact a simpler method of chromatographic purification can be employed. As the acidic A subunit (crotapotin) is vital to the specific activity of the molecule, purification by ion exchange on QAE sepharose selects for this subunit. Tris-hydroxy-amidomethane (Tris) or phosphate buffers at pH's above 6.5 can be employed in association with 0.15M NaCl to aide crotoxin solubility. Elution with NaCl or KCl gradients up to 1.5M are preferred. The crotoxin molecule elutes toward the end of the gradient, distinctly separated from potential contaminants such as crotamine. The resulting crotoxin peak is dialysed against 0.15M NaCl where it is ready for dilution to the correct concentration.

### EXAMPLES

### Example 1

Toxicity Assay in Mice

The potency of crotoxin is most easily determined by assessing the toxicity of the preparation in mice. Mice are sensitive to the actions of many venoms particularly to that of snakes. The reported LD50 of pure crotoxin in mice is 75 mcg.Kg⁻¹. The injection of 150 mcg of crotoxin into a 20 g mouse will induce death within 2 hours. If the animal survives overnight it is accepted that the material lost its lethal effects and is therefore suspect.

### Example 2

Antiviral Assay 1

Fresh human blood was obtained commercially from Interstate Blood Bank, Inc. (Memphis, Term.). The low-passage, lymphotropic clinical isolates HIV-1_{WEJO} and HIV-1_{TEKI} were obtained from pediatric patients attending the AIDS Clinic at the University of Alabama at Birmingham. The clinical isolate HIV-1_{TEKI} has been typed as non-syncytium inducing (NSI) in MT-2 cells, while the HIV-1_{WEJO} isolate has been typed as syncytium inducing (SI). The SI and NSI phenotypes have been correlated with lymphocyte and monocyte tropism, respectively, and these viruses have been found to favor the corresponding coreceptors for infection. Pre-titered aliquots of HIV-1_{WEJO} and HIV-1_{TEKI} were removed from the freezer (−80° C.) and thawed rapidly to room temperature in a biological safety cabinet immediately before use. Phytohemagglutinin (PHA-P) was obtained from Sigma (St. Louis, Mo.) and recombinant IL-2 was obtained from R&D Systems Inc. (Minneapolis, Minn.).

b. Anti-HIV Efficacy Evaluation in Fresh Human PBMCs

Fresh human PBMCs were isolated from screened donors, seronegative for HIV and HBV. Cells were pelleted/washed 2-3 times by low speed centrifugation and resuspension in PBS to remove contaminating platelets. The Leukophoresed blood was then diluted 1:1 with Dulbecco's phosphate buffered saline (PBS) and layered over 14 mL of Ficoll-Hypaque density gradient (Lymphocyte Separation Medium, Cell Grow #85-072-CL, density 1.078+/−0.002 gm/ml) in a 50 mL centrifuge tube and then centrifuged for 30 minutes at 600×g. Banded PBMCs were gently aspirated from the resulting interface and subsequently washed 2× with PBS by low speed centrifugation. After the final wash, cells were enumerated by trypan blue exclusion and re-suspended at 1×10⁷ cells/mL in RPMI 1640 supplemented with 15% Fetal Bovine Serum (FBS), 2 mM L-glutamine, 4 μg/mL PHA-P. The cells were allowed to incubate for 48-72 hours at 37° C. After incubation, PBMCs were centrifuged and resuspended in RPMI 1640 with 15% FBS, 2 mM L-glutamine, 100 U/mL penicillin, 100 μg/mL streptomycin, 10 μg/mL gentamycin, and 20 U/mL recombinant human IL-2. PBMCs were maintained in this medium at a concentration of 1-2×10⁶ cells/mL with biweekly medium changes until used in the assay protocol. Monocytes were depleted from the culture as the result of adherence to the tissue culture flask.

c. Reverse Transcriptase Activity Assay

A microtiter plate-based reverse transcriptase (RT) reaction was utilized (Buckheit et al., AIDS Research and Human Retroviruses 7:295-302, 1991). Tritiated thymidine triphosphate (³H-TTP, 80 Ci/mmol, NEN) was received in 1:1 dH₂O:Ethanol at 1 mCi/ml. Poly rA:oligo dT template:primer (Pharmacia) was prepared as a stock solution by combining 150 μl poly rA (20 mg/ml) with 0.5 ml oligo dT (20 units/ml) and 5.35 ml sterile dH₂O followed by aliquoting (1.0 ml) and storage at −20° C. The RT reaction buffer was prepared fresh on a daily basis and consisted of 125 μl 1.0 M EGTA, 125 μl dH₂O, 125 μl 20% Triton X100, 50 μl 1.0 M Tris (pH 7.4), 50 μl 1.0 M DTT, and 40 μl 1.0 M MgCl₂. The final reaction mixture was prepared by combining 1 part ³H-TTP, 4 parts dH₂O, 2.5 parts poly rA:oligo dT stock and 2.5 parts reaction buffer. Ten microliters of this reaction mixture was placed in a round bottom microtiter plate and 15 μl of virus-containing supernatant was added and mixed. The plate was incubated at 37° C. for 60 minutes. Following incubation, the reaction volume was spotted onto DE81 filter-mats (Wallac), washed 5 times for 5 minutes each in a 5% sodium phosphate buffer or 2×SSC (Life Technologies), 2 times for 1 minute each in distilled water, 2 times for 1 minute each in 70% ethanol, and then dried. Incorporated radioactivity (counts per minute, CPM) was quantified using standard liquid scintillation techniques.

### Example 3

Antiviral Assay 2.

The antiviral activity was repeated in a second independent laboratory as follows; PBMCs from fresh, HIV-1 non-infected buffy coat cells obtained from healthy donors at local blood banks were purified by the Ficoll method. The buffy coat cells were maintained at room temperature until centrifugation. Purified PBMC were re-suspended at 1E6-3E6 cells/mL RPMI medium supplemented with 10% human AB serum and immediately treated with 5 mcg PHA/mL suspension. Two to three days later, cells were counted and used for examination of infection. As a standard procedure, cells were incubated in propagation media, consisting of RPMI media supplemented with 10% human AB serum and 50 units IL2/mL, at a density of 6E6 cells per mL and incubated with 200-1000 TCID₅₀ HIV-1/mL×10E6 PBMC. Infection was allowed for 2 hours at 37° C. and the unbound virus was washed away by two washes with propagation media. 200,000 cells were suspended in 180 uL of propagation media and placed in 96 well plates (U bottom). Twenty mcL of a 10× stock of the corresponding dilution of the drug was added to each well. Infections were performed in triplicate and controls containing 1 microM AZT were run in parallel as controls to confirm the validity of the assay. The cultures were incubated at 37° C. for 4 days. At that time, 90 uL of media was removed and replaced with 100 mcL of propagation media containing the corresponding dilution of drug. The amount of p24 accumulated in the culture was estimated 3 days later (7 days post infection) with a Becton-Dickenson p24 ELISA. Routinely, a few samples were chosen and 10E-2 to 10E-4 dilutions of culture supernatant were prepared to estimate the linearity of the assay.

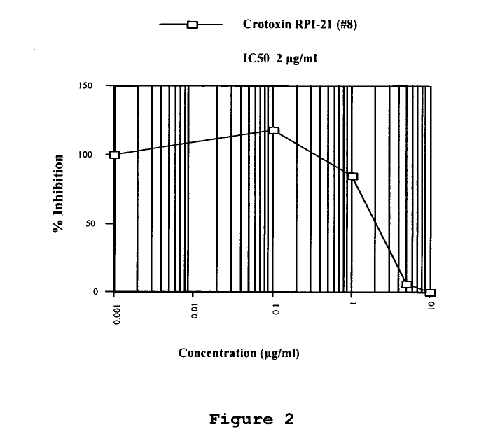

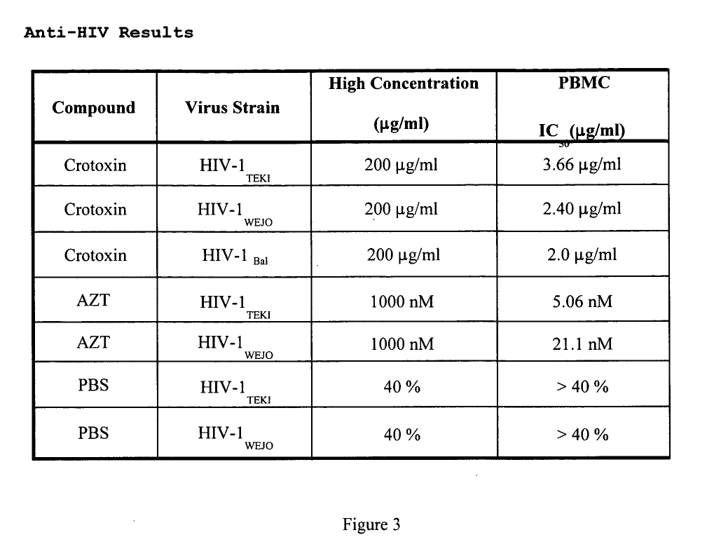

As seen in FIGS. 2 and 3, Crotoxin was active in these antiviral assays and had IC₅₀ values of ranging from 3.66 μg/ml and 2.0 μg/ml versus HIV-1_{Teki}, HIV-1_{Teki} and HIV-1_{Bal}. As AZT was employed as a model positive control it may be helpful to consider the activity of crotoxin in direct comparison to it. In the molar range, Crotoxin was effective from 0.153 nM down to 0.083 nM. This would make crotoxin 60 to 137 times more effective than AZT. Furthermore, it would suggest that crotoxin was 3-5 times more potent than those described for the typical cobra venom phospholipase though this variance could be due to variable assay parameters. As a neurotoxin, crotoxin could prevent the progress of AIDS to AIDS-related dementia due to its ability to block neuronal receptors including the nicotinic acetylcholine receptor.

These data confirm that the bi-partite toxin could serve as a therapeutic for HIV infection especially with respect to new infections as the Bal isotype employs the CCR5 chemokine receptor as its portal into the macrophages, a critical step in new infections. Furthermore, a crotoxin-based lotion or lubricant could be employed alone or along with spermicides to block the initiating infection during heterosexual intercourse where CCR5-using isolates gain entry into the host. In this format the crotoxin may not be required to be isolated from the venom prior to incorporation into the topical product. Topical application of Crotalus durissus venom causes no ill effects on intact skin surfaces supporting this specification.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

What is claimed is:

1. A pharmaceutical composition comprising a therapeutically effective amount of toxin from the group including crotoxin and mojavetoxin having corresponding biological activity and a pharmaceutically acceptable carrier for use in inhibiting infection by retroviruses.

2. The composition of claim 1 wherein the crotoxin is obtained from the snake Crotalus durissus terrificus and the mojavetoxin is obtained from the snake Crotalus scutulatus scutulatus.

3. The composition of claim 1 for parenteral (intravenous, intramuscular or subcutaneous) administration delivering between 0.13 mcg.kg⁻¹ of body weight per day up to a maximum of 40 mcg. kg⁻¹ of body weight per day.

4. The composition of claim 1 for topical administration comprising substantially between 6 mcg and 1 mg of toxin per gram of base.

5. The composition of claim 5 in which the toxin is crotoxin at a concentration of 100-200 mcg per gram of base.

6. A method of treatment of retroviral infection in one of the human and the animal body comprising administering an effective amount of a composition comprising a toxin from the group including crotoxin and mojavetoxin having corresponding biological activity.

7. The method of claim 1 wherein the retroviral infection is selected from a group comprising human immunodeficiency virus (HIV), simian immunodeficiency virus (SIV), feline immunodeficiency virus (FIV), bovine immunodeficiency virus (BIV) and equine acquired immunodeficiency virus (EAIV).
